(19) United States
(12) Patent Application Publication
(10) Pub. No.: US 2014/0375297 A1
(43) Pub. Date: Dec. 25, 2014

(54) Ionic Fluid Detection System For Absorbent Articles

(71) Applicants: Allen R. Geiger, Las Cruces, NM (US); Edward J. Burlbaw, Las Cruces, NM (US)

(72) Inventors: Allen R. Geiger, Las Cruces, NM (US); Edward J. Burlbaw, Las Cruces, NM (US)

(21) Appl. No.: 13/922,240

(22) Filed: Jun. 19, 2013

(51) Int. Cl. A61F 13/42; G01N 27/00

(52) U.S. Cl. CPC A61F 13/42; G01N 27/00
USPC 324/71.1; 604/361

(57) ABSTRACT

An ionic fluid detection system for absorbent articles has a radiofrequency receiver operable to receive a first radiofrequency signal, a radiofrequency emitter connected to the receiver and operable to emit a second radiofrequency signal, the emitter being responsive to the receiver to emit a second radiofrequency signal when the receiver receives the first radiofrequency signal, the responsiveness of the emitter being enabled when the sensor is in a first wetness condition selected from the group of conditions including “wet” and “dry” and being disabled when in the other wetness condition. The second radiofrequency signal may have a different frequency and wavelength than the first radiofrequency signal. The radiofrequency receiver may be electrically connected to the radiofrequency emitter by a diode to form a resonant antennae pair. There may be a ground plane and a nonconductive, ionic fluid wicking material connected to the ground plane and the receiver and/or the emitter.

### FIELD OF THE INVENTION

The present invention relates to disposable absorbent articles, and more particularly to systems for detecting the presence of ionic fluids in a disposable absorbent article, such as a diaper.

### BACKGROUND OF THE INVENTION

Diapers are a type of underwear suitable for receiving and containing the bodily excretions of a wearer. When diapers become soiled, they require changing. The changing procedure is often performed by a second person, such as a parent or caregiver. Failure to change soiled diapers frequently can result in skin problems such as irritant diaper dermatitis, commonly referred to as diaper rash. Irritant diaper dermatitis develops when skin is exposed to prolonged wetness and increased skin pH caused by urine and feces, which result in the outermost layer of the skin breaking down.

In order to prevent irritant diaper dermatitis, the diaper must be frequently checked for wetness.

This typically involves the placing of a finger inside the diaper or physically feeling the diaper. The process is time-consuming, unpleasant, and in the case of attendant care for bedridden adults, is costly in terms of time and secondary costs associated with any skin issues that develop.

Modern disposable baby diapers and incontinence products have a layered construction, which allows the transfer and distribution of urine to an absorbent core structure where it is locked in. Basic layers are an outer shell of breathable polyethylene film or a nonwoven and film composite, which prevents wetness and soil transfer, an inner absorbent layer of a mixture of air-laid paper and superabsorbent polymers for wetness, and a layer nearest the skin of nonwoven material, with a distribution layer directly beneath, which transfers wetness to the absorbent layer.

Other common features of disposable diapers include one or more pairs of either adhesive or hook and loop fastener tapes to keep the diaper securely fastened. Some diapers have tapes which are refastenable to allow adjusting of fit or reapplication after inspection. Elasticized fabric single and double gussets around the leg and waist areas aid in fitting and in containing urine or stool which has not been absorbed. Most materials in the diaper are held together with the use of a hot melt adhesive which is applied in spray form or multi lines, an elastic hot melt is also used to help with pad integrity when the diaper is wet.

There are numerous prior art diapers that seek to communicate the wet/dry status of the diaper to a second person using a visual indicator. Many techniques are known for communicating the presence of wetness for this purpose, including materials that change color in response to temperature change, pH change, and/or the presence of water. However, all of these techniques require the second person to be within visual range of the status indicator, to make the effort to look at the status indicator frequently, and if clothing is worn over the diaper, to remove the clothing to uncover the status indicator for viewing and to replace the clothing if the diaper is not wet. These requirements make a visual wetness indicator more of a gimmick rather than a highly useful tool for the caregiver or parent.

Therefore, a need exists for a new and improved ionic fluid detection system for absorbent articles that can communicate when the absorbent article is wet to a second person at a distance without requiring the attention of the second person until the absorbent article is wet. In this regard, the various embodiments of the present invention substantially fulfill at least some of these needs. In this respect, the ionic fluid detection system for absorbent articles according to the present invention substantially departs from the conventional concepts and designs of the prior art, and in doing so provides an apparatus primarily developed for the purpose of detecting and communicating to a second person when absorbent article is wet without requiring the attention of the second person until the absorbent article is wet.

### SUMMARY OF THE INVENTION

The present invention provides an improved ionic fluid detection system for absorbent articles, and overcomes the above-mentioned disadvantages and drawbacks of the prior art. As such, the general purpose of the present invention, which will be described subsequently in greater detail, is to provide an improved ionic fluid detection system for absorbent articles that has all the advantages of the prior art mentioned above.

To attain this, the preferred embodiment of the present invention essentially comprises a radiofrequency receiver operable to receive a first radiofrequency signal, a radiofrequency emitter connected to the receiver and operable to emit a second radiofrequency signal, the emitter being responsive to the receiver to emit a second radiofrequency signal when the receiver receives the first radiofrequency signal, the responsiveness of the emitter being enabled when the sensor is in a first wetness condition selected from the group of conditions including “wet” and “dry” and being disabled when in the other wetness condition. The second radiofrequency signal may have a different frequency and wavelength than the first radiofrequency signal. The radiofrequency receiver may be electrically connected to the radiofrequency emitter by a diode to form a resonant antennae pair. There may be a ground plane and a nonconductive, ionic fluid wicking material connected to the ground plane and at least one of the group consisting of the radiofrequency receiver and the radiofrequency emitter. There are, of course, additional features of the invention that will be described hereinafter and which will form the subject matter of the claims attached.

There has thus been outlined, rather broadly, the more important features of the invention in order that the detailed description thereof that follows may be better understood and in order that the present contribution to the art may be better appreciated.

### BRIEF DESCRIPTION OF THE DRAWINGS

FIG. 1 is a top plan view of the current embodiment of an ionic fluid detection system constructed in accordance with the principles of the present invention.

FIG. 1A is an enlarged view of the ionic fluid sensor of FIG. 1.

FIG. 2 is a schematic of the ionic fluid detection system of the present invention.

FIG. 3A is a schematic of a first alternative embodiment of the ionic fluid detection sensor of the present invention that incorporates a pull tab-style activation strip.

FIG. 3B is a schematic of a second alternative embodiment of the ionic fluid detection system of the present invention that incorporates a break tab-style activation strip.

FIG. 4 is a top plan view of a disposable diaper, with the upper layers partially cut away, that includes the ionic fluid sensor of the present invention.

FIG. 5 is a front perspective view of the disposable diaper of FIG. 4.

FIG. 6 is a back perspective view of the disposable diaper of FIG. 4.

FIG. 7 is a cross-sectional view of the disposable diaper shown in FIG. 4.

FIG. 8 is a perspective view of a disposable pant-type garment constructed in accordance with the principles of the present invention.

FIG. 9 is a top plan view of a disposable bandage that includes the ionic fluid sensor of the present invention.

FIG. 10 is a cross-sectional view of the disposable bandage shown in FIG. 9.

FIG. 11 is a top plan view of a disposable bed pad, with the upper layer partially cutaway, that includes the ionic fluid sensor of the present invention.

The same reference numerals refer to the same parts throughout the various figures.

### DESCRIPTION OF THE CURRENT EMBODIMENT

An embodiment of the ionic fluid detection system of the present invention is shown and generally designated by the reference numeral 100.

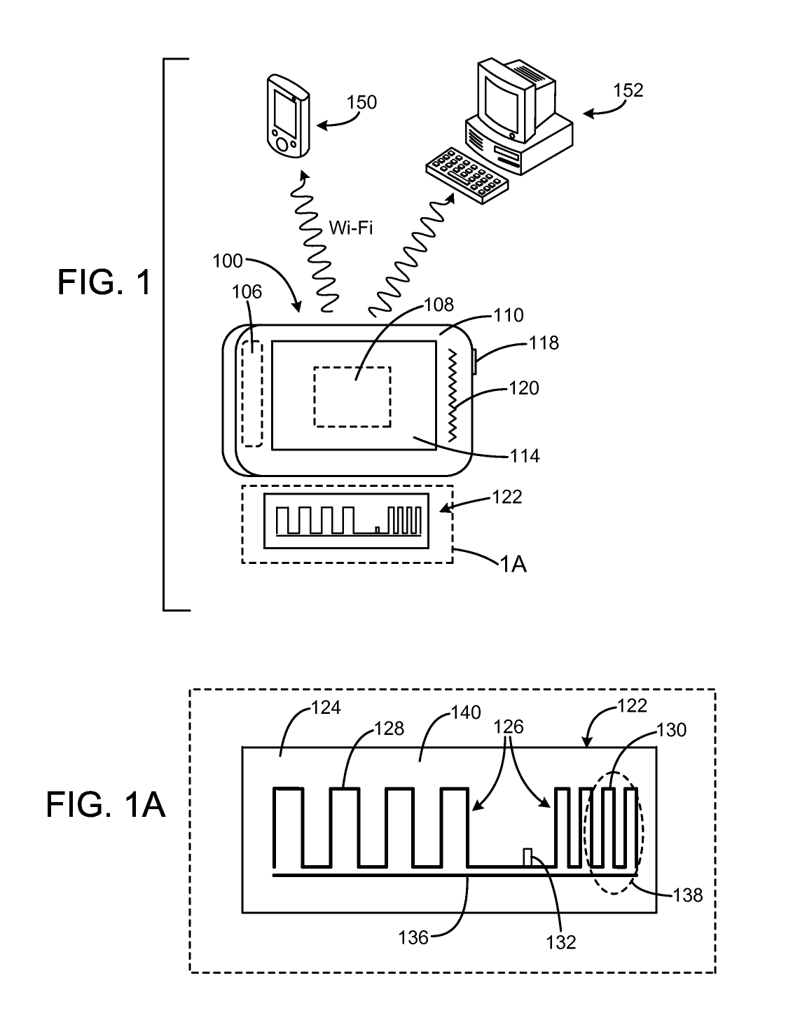

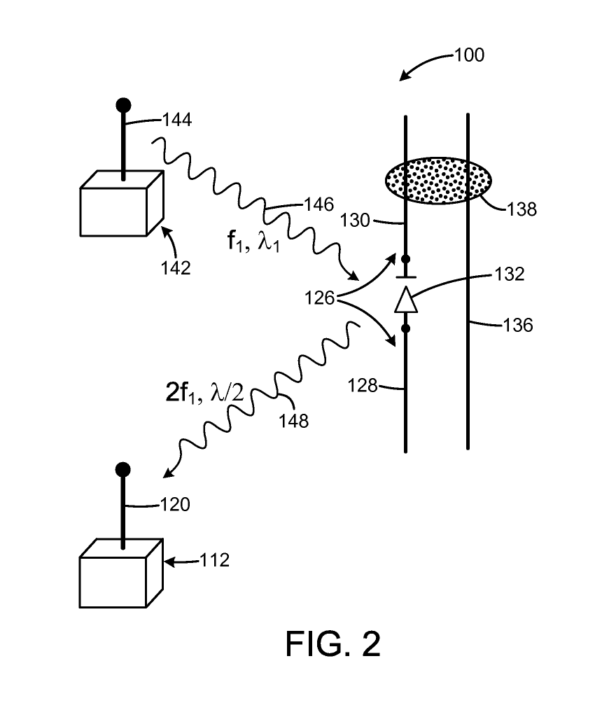

FIGS. 1 and 2 illustrate the ionic fluid detection system 100 of the present invention. More particularly, the system includes a radiofrequency transmitter 142, an ionic fluid sensor 122, and a radiofrequency receiver/reader) 112. The system uses resonant antenna architecture to detect the presence of an ionic fluid.

The sensor includes a resonant antennae pair 126 that consists of a receiver antenna 128 that is connected to a transmitter antenna 130 by a diode 132. The function of the diode is to allow an electric current to pass from the receiver antenna to the transmitter antenna, while blocking current in the opposite direction from the transmitter antenna to the receiver antenna. The diode is a non-linear device and produces all harmonics of the fundamental frequency. The transmitter antenna is designed to be resonant with one of those harmonics; i.e., 2^{nd}, 3^{rd}, 4^{th}, etc. The length of the transmitter antenna is tuned to an integer multiple of the receiver frequency. The frequencies emitted by the transmitter antenna are higher (shorter wavelength) than those received by the receiver antenna. A ground plane 136 is placed near the resonant pair antenna. The antennae and ground plane can be linear, spiral, repeating, rectangular, or fractal geometries. A sensor pad 138 is placed across at least a portion of the antennae pair and the ground plane. The sensor pad may be cotton or any other suitable wicking material. This wicking material can also be used to move fluids to the sensor so the antennae pair can be remote to the actual measurement site (i.e. the antennae can be located in the elastic band, with a thin wicking channel to the diaper crotch).

The antennae 126 can be constructed using conventional metallic materials, or they may be constructed of any conductive material (conductive paints, carbon, graphite, carbon nanotubes, or graphene). These materials allow the device to be constructed using inkjet printing techniques to lower production costs. The diode 132 can be a solder, glue or press-on device, or may also be ink jet printed.

The antennae 126 can be fabricated on any suitable substrate material 140, including a flexible circuit board, or any insulating material, including paper. An advantage of using a paper substrate with a carbon-based antennae is that the sensor 122 is completely disposable, environmentally benign, and very low cost to fabricate.

The radiofrequency transmitter 142 includes an antenna 144. The transmitter broadcasts radio waves 146 continually or intermittently at a suitable interval at a specified frequency. Although any frequency can be used, a frequency within the 900 MHz band is preferred for its suitability for local communications and its regulatory allocation to industrial, scientific, and medical devices as well as secondary amateur use.

The radiofrequency receiver receiver/reader 112 includes a processor 108, a body 110, a display 114, a battery 116, an on/off switch 118, and one or more antennas 120. The processor in combination with the antenna(s) acts as a transceiver: as a receiver, the reader is tuned to detect one or more radio waves 148 having wavelengths that are a multiple of the radio waves 146 broadcast by the transmitter 142; as a transmitter, the reader acts as a wireless local area network product based on the Institute of Electrical and Electronics Engineers' 802.11 standards, commonly known as Wi-Fi. The Wi-Fi compliant radio waves enable the reader to exchange data wirelessly with a smart phone 150 and/or monitoring station enabled by personal computer 152.

A. Geiger et al. 5

In use, the receiver antenna 128 is stimulated by radio waves 146 having a frequency fl and a wavelength λ1 broadcast from the transmitter 142 (for example, 900 MHz). The stimulated receiver antenna tries to resonate with the transmitter antenna 130. The transmitter antenna then radiates a radio wave at a wavelength λ2 that is some multiple of the wavelength λ1 received by the receiver antenna. In our example, with a 900 MHz receiving antenna, the radio waves 148 transmitted by the sensor 122 will be at a frequency f2 (to a shorter wavelength and higher frequency) of 1.8 GHz, in this case the second harmonic. This output wavelength is picked up by the tuned receiver/reader 112 via antenna 120, and then processed by the processor 108. As long as the receiver/reader receives output from the sensor, the processor causes the receiver/reader to transmit Wi-Fi signals indicating the sensor is dry.

In the event the sensor pad 138 is wetted by an ionic fluid, the sensor pad becomes conductive and shorts out the resonant antennae pair 126 to the ground plane 136. When that occurs, the sensor 122 stops transmitting. The sensor pad can be located anywhere along the resonant antennae pair and the ground plane because the fluidic short-circuit at any point will disrupt the radiofrequency transmission. In addition, when paper is used as the substrate 140 of the sensor 122, the sensor can function by the paper dissolving in response to wetting, thereby breaking the resonant antennae pair and disrupting the radiofrequency transmission.

Responsive to the processor detecting the loss of the sensor transmission, the processor causes the receiver/reader 112 to transmit Wi-Fi signals indicating the sensor is wet. The Wi-Fi signals can be received by a suitable Wi-Fi capable product, such as a smartphone 150 or a personal computer 152. The Wi-Fi capable product can then generate a user perceptible signal, such as an audible, visual, and/or tactile signal, to alert the user that the sensor is wet and action is required.

In operation, the sensors may be designed for local readers operating at short range. In this case, the power levels of the transmitter 142 and receiver/reader 112 are set for operation from a location in close proximity to the sensor where, such as a bed rail (babies, nursing home patients), or from the pocket of a health care provider. The reader/receiver can be carried and used as a wand to check for wetness of a proximate sensor. The receiver/reader may also be used with more radiofrequency power to monitor a group of people in a room. During operation, the transmitter and receiver/reader operate intermittently to extend battery life and reduce radiofrequency exposure. In the case of babies and chronic care patients, an interrogation once every 5 minutes could be adequate. In the case where there are multiple diapers in use, and there is overlap of signals from the reader, the wet diaper can be found by reducing the radiofrequency signal output and using the reader as a wand in close proximity to each diaper to check for wetness. This is the lowest cost approach.

The sensor antennae could be modified using a programmable inductor, resistor, or capacitor attached to the antennae. The inductor, resistor, or capacitor, which could be composed of a magnetic or conductive ink, is programmed by the reader, which produces a magnetic pattern in the ink. This ink strip acts like a variable or programmable inductor, resistor, or capacitor on the transmitting antennae, causing a slight frequency shift to provide each diaper with a differentiated frequency to facilitate identification.

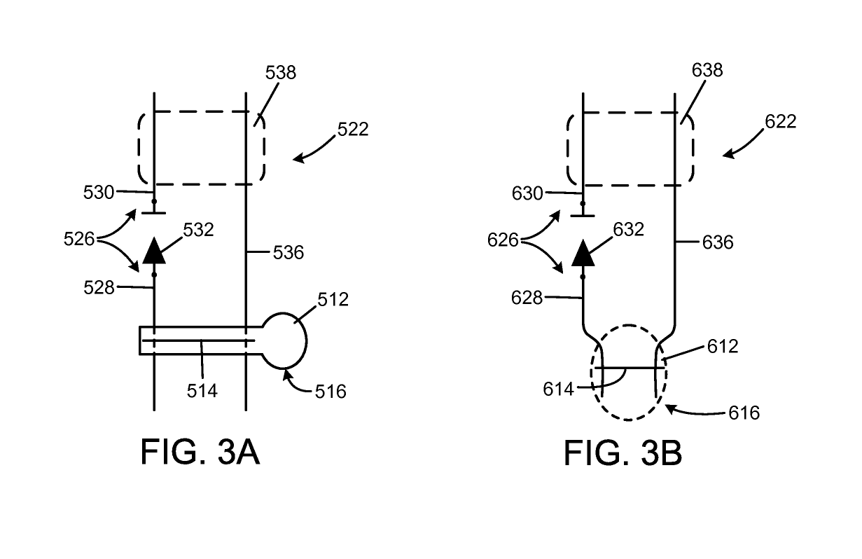

FIGS. 3A & 3B illustrate alternative embodiments of the ionic fluid detection sensor 522 and 622 of the present invention. More particularly, the sensors 522, 622 include a resonant antennae pair 526, 626 that consists of a receiver antenna 528, 628 that is connected to a transmitter antenna 530, 630 by a diode 532, 632. In order to avoid detection of unused diapers in the vicinity, each sensor has an activation strip 516, 616 that “turns on” the antennae. This is accomplished by means of deactivating an antenna “short” 514, 614. This short is a conductive material laid across the antennae. Activation is achieved by removing this short by means of a pull tab 512 (FIG. 3A), which removes the conductive strip 514, or with an activation “button” 614 (FIG. 3B), which breaks the shorting material 614. Activation is performed only immediately prior to placing the diaper on a wearer.

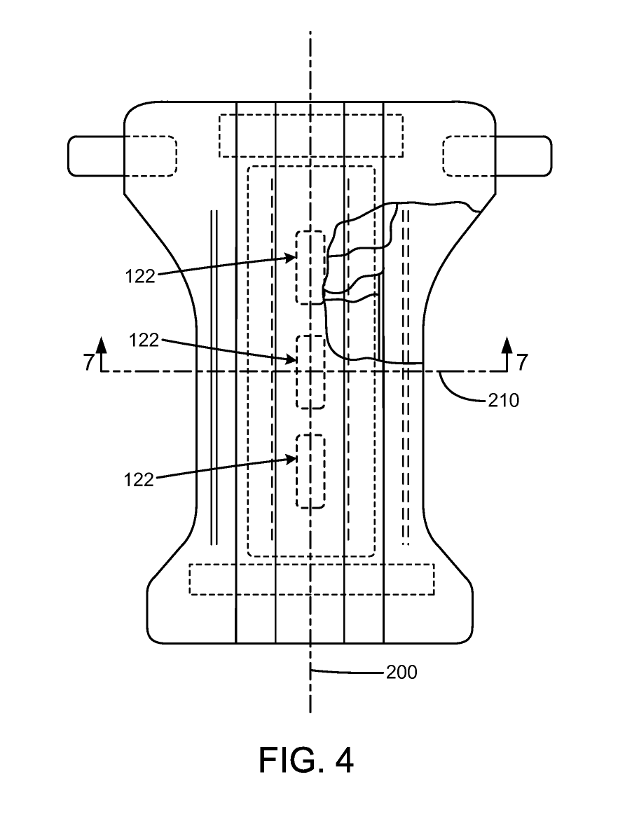

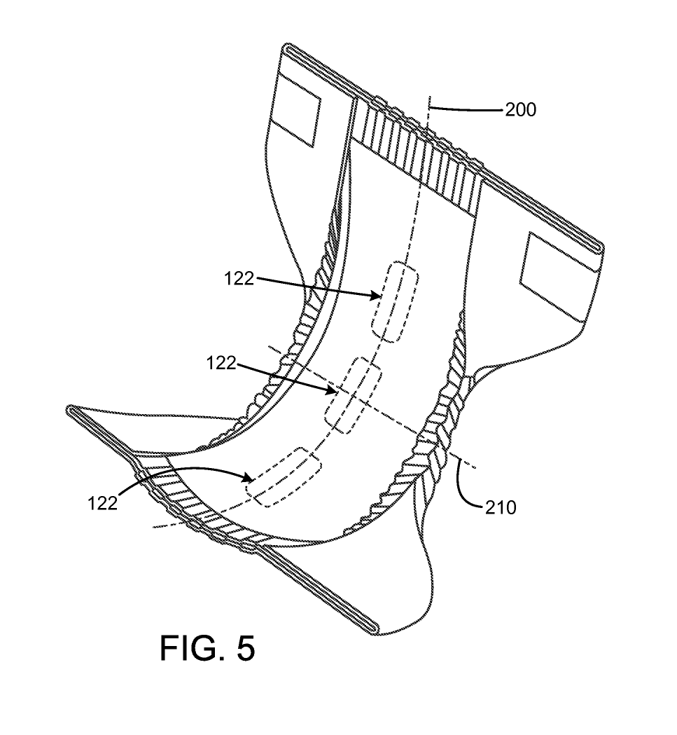

FIGS. 4-7 illustrate an absorbent article, specifically a disposable diaper 20, which includes the ionic fluid sensor 122 of the present invention, which makes the diaper operable for use with the radiofrequency transmitter 142 and the radiofrequency receiver/reader 112 of the present invention. More particularly, in FIG. 4, the diaper is shown in its flat out, uncontracted state (i.e., without elastic-induced contraction). Portions of the structure are cut away to more clearly show the underlying structure of the diaper 20. The portion of the diaper that contacts a wearer is facing the viewer.

The chassis 22 of the diaper in FIG. 4 comprises the main body of the diaper. The chassis comprises an outer covering including a liquid pervious topsheet 24 and/or a liquid impervious backsheet 26. The chassis may also include most or all of the absorbent core 28 encased between the topsheet and the backsheet. The chassis preferably further includes side panels 30, leg cuffs 32, and a waist feature 34. The leg cuffs and the waist feature typically comprise elastic members 33. One end portion of the diaper is configured as the front waist region 36 of the diaper. The opposite end portion is configured as the rear waist region 38 of the diaper. An intermediate portion of the diaper is configured as the crotch region 37, which extends longitudinally between the front and rear waist regions 36 and 38. The crotch region is that portion of the diaper which, when the diaper is worn, is generally positioned between the wearer's legs.

The waist regions may include a fastening system comprising fastening members 40 preferably attached to the rear waist region 38 and a landing zone 42 attached to the front waist region. The diaper has a longitudinal axis 200 and a transverse axis 210. The periphery of the diaper is defined by the outer edges of the diaper in which the longitudinal edges 44 run generally parallel to the longitudinal axis of the diaper and the end edges 46 run generally parallel to the transverse axis 110 of the diaper.

The backsheet 26 prevents the exudates absorbed by the absorbent core 28 and contained within the diaper 20 from soiling other external articles that may contact the diaper, such as bed sheets and undergarments. In preferred embodiments, the backsheet is substantially impervious to liquids (e.g., urine). Suitable backsheet materials may include breathable materials that permit vapors to escape from the diaper while still preventing exudates from passing through the backsheet.

The absorbent core 28 generally is positioned between the topsheet 24 and the backsheet 26.

The absorbent core may comprise any absorbent material that is generally compressible, conformable, non-irritating to the wearer's skin, and capable of absorbing and retaining liquids such as urine and other certain body exudates. The absorbent core 28 may comprise a wide variety of liquid-absorbent materials commonly used in disposable diapers and other absorbent articles.

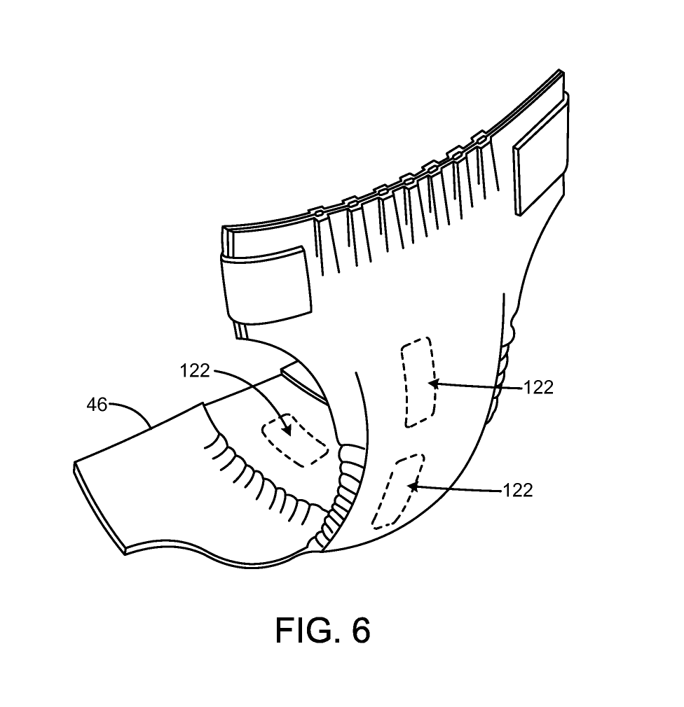

One or more ionic fluid (i.e., urine) sensors 122 of the present invention are encased between the topsheet and the backsheet. The sensors can be placed in any portion of the diaper, but preferably within the crotch region 37 along the longitudinal axis 200 so as to detect wetness as soon as possible. The location of the sensors may vary depending upon the gender and/or age of the intended wearer of the diaper. As will be discussed further in the description of FIG. 6, the sensors may be placed at any height within the interior volume of the diaper defined by the topsheet and backsheet, ranging from abutting the underside of the topsheet to abutting the top side of the backsheet.

The diaper 20 may also include such other features as are known in the art including front and rear ear panels, waist cap features, elastics and the like to provide better fit, containment and aesthetic characteristics.

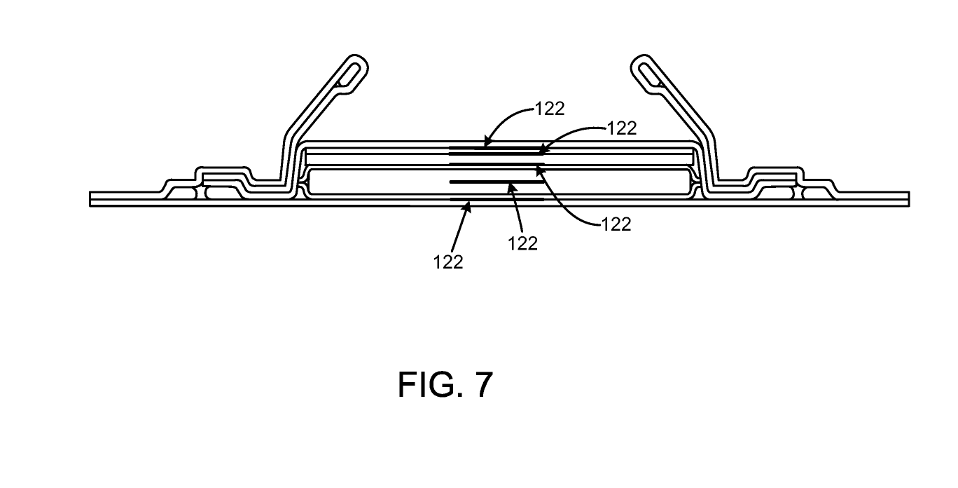

In order to keep the diaper 20 in place about the wearer, the waist regions 36 and 38 may include a fastening system comprising fastening members 40 preferably attached to the rear waist region. In a preferred embodiment the fastening system further comprises a landing zone 42 attached to the front waist region 36. The fastening member is attached to the front waist region, preferably to the landing zone to form leg openings and an article waist. Diapers 20 according to the present invention may be provided with a re-closable fastening system or may alternatively be provided in the form of a pant-type diaper 220 (shown in FIG. 7).

FIG. 7 shows a cross-sectional view of FIG. 3 taken in the transverse axis 210. Starting from the wearer facing side, the diaper comprises the topsheet 24, the components of the absorbent core 28, and the backsheet 26. The absorbent core preferably comprises an acquisition system 50, which comprises an upper acquisition layer 52 facing towards the wearer and a lower acquisition layer 54. The acquisition layer preferably is in direct contact with the storage layer 60.

The storage layer 60 is preferably wrapped by a core wrap material. In one preferred embodiment the core wrap material comprises a top layer 56 and a bottom layer 58. The storage layer 60 typically comprises fibrous materials, mixed with superabsorbent, absorbent gelling materials.

The ionic fluid sensors 122 of the present invention can reside at any height between the topsheet 24 and the backsheet 26. In the embodiment shown in FIG. 6, sensors are placed between the backsheet and the upper acquisition layer 52, between the upper acquisition layer and the lower acquisition layer 54, between the lower acquisition layer and the top side of the absorbent core 28, within the absorbent core 28, and between the underside of the absorbent core and the backsheet. By positioning sensors at different heights of the diaper, with each sensor transmitting at a different wavelength, the processor 108 in the receiver/reader 112 can determine how full of liquid the diaper 20 is based on the number of sensors that are detected. As additional sensor signals are lost, the processor can cause the receiver/reader to transmit Wi-Fi signals that cause the smartphone 150 or personal computer 152 to create increasingly urgent user perceptible signals as the diaper appears to be reaching capacity and/or after a set period of time has passed since the first detection of wetness occurred. Alternatively, sensor height can also be used to adjust sensitivity to wetness. For example, sensors could be positioned only deeper within the diaper so that the storage capacity of the diaper is more fully utilized before a user perceptible signal that the diaper is wet is generated.

In a situation where multiple diaper wearers are being monitored, each wearer could have sensor(s) that transmit unique frequencies that identify the wearer to the smartphone or personal computer. The smart phone or personal computer could track which wearers have been wet the longest and which wearers are the wettest so that a parent or caregiver would know the optimal order in which to change the wearers' diapers.

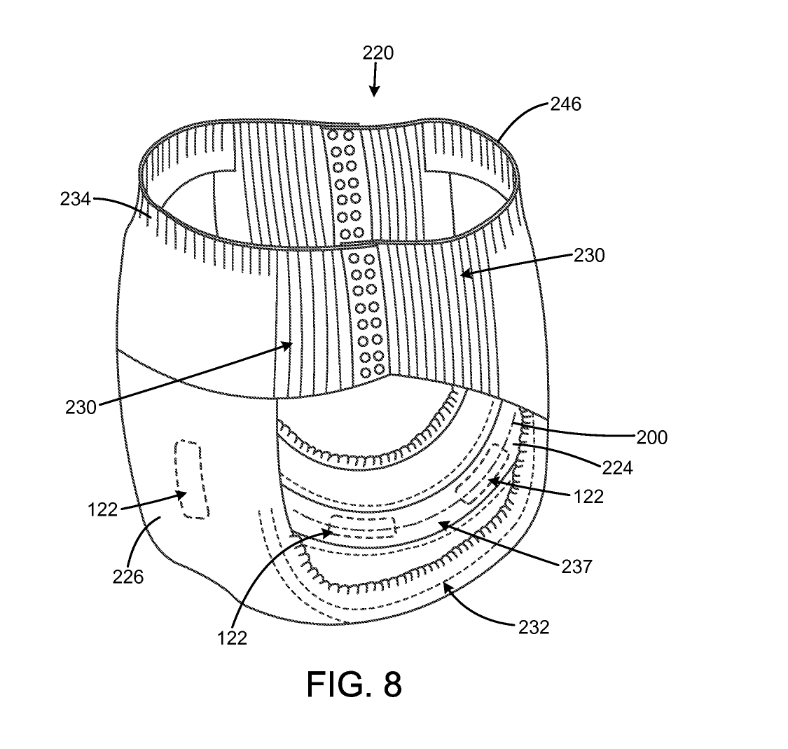

FIG. 8 illustrates an absorbent article, specifically a pant-type diaper 220 that is preformed by the manufacturer, which includes the ionic fluid sensor 122 of the present invention, which makes the diaper operable for use with the radiofrequency transmitter 142 and the radiofrequency receiver/reader 112 of the present invention. More particularly, the pant-type diaper 220 comprises a chassis 222 with a topsheet 224 and backsheet 226, side panels 230, leg cuffs 232, a waist feature 234, and end edges 246. These features have the same functions as the corresponding features of the diaper 20, so no further description will be provided. One or more ionic fluid (i.e., urine) sensors 122 of the present invention are encased between the topsheet and the backsheet. The sensors can be placed in any portion of the pant-type diaper, but preferably within the crotch region 237 along the longitudinal axis 200 so as to detect wetness as soon as possible. The location of the sensors may vary depending upon the gender and/or age of the intended wearer of the diaper. The sensors may be placed at any height within the interior volume of the diaper defined by the topsheet and backsheet, ranging from abutting the underside of the topsheet to abutting the top side of the backsheet.

The terms “pant” or “pant-type diaper,” as used herein, refers to disposable garments having a waist opening and leg openings designed for infant or adult wearers. A pant may be placed in position on the wearer by inserting the wearer's legs into the leg openings and sliding the pant into position about the wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the term “pant” is used herein, pants are also commonly referred to as “closed diapers,” “prefastened diapers,” “pull-on diapers,” “training pants,” “swim pants,” and “diaper-pants.”

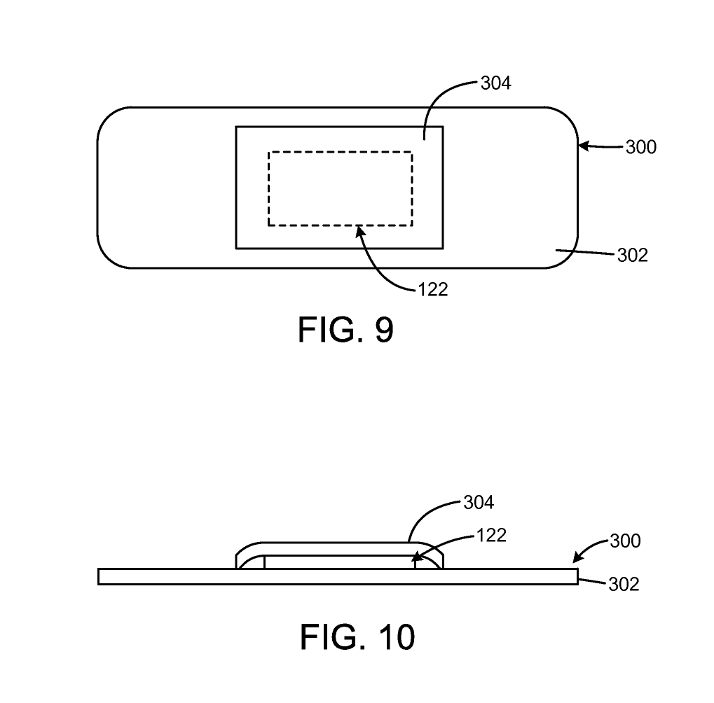

FIGS. 9 and 10 illustrate an absorbent article, specifically a bandage 300, which includes the ionic fluid sensor 122 of the present invention, which makes the bandage operable for use with the radiofrequency transmitter 142 and the radiofrequency receiver/reader 112 of the present invention. More particularly, the portion of the bandage that contacts a wearer is facing the viewer. The bandage has an absorbent layer 304 that is attached to an adhesive layer 302. One or more ionic fluid (i.e., wound exudate) sensors 122 of the present invention are encased between the absorbent layer and the adhesive layer. The sensors can be placed in any portion of the bandage, but preferably abutting the underside or placed within the absorbent layer so as to detect wetness as soon as possible. The location of the sensors may vary depending upon the type of bandage. The sensors may be placed at any height within the interior volume of the bandage defined by the absorbent layer and the adhesive layer. The sensor pad can be modified by adding chemical sensors to detect infectious by-products (NH4 gas, body break-down materials, etc.) as well as ionic fluids.

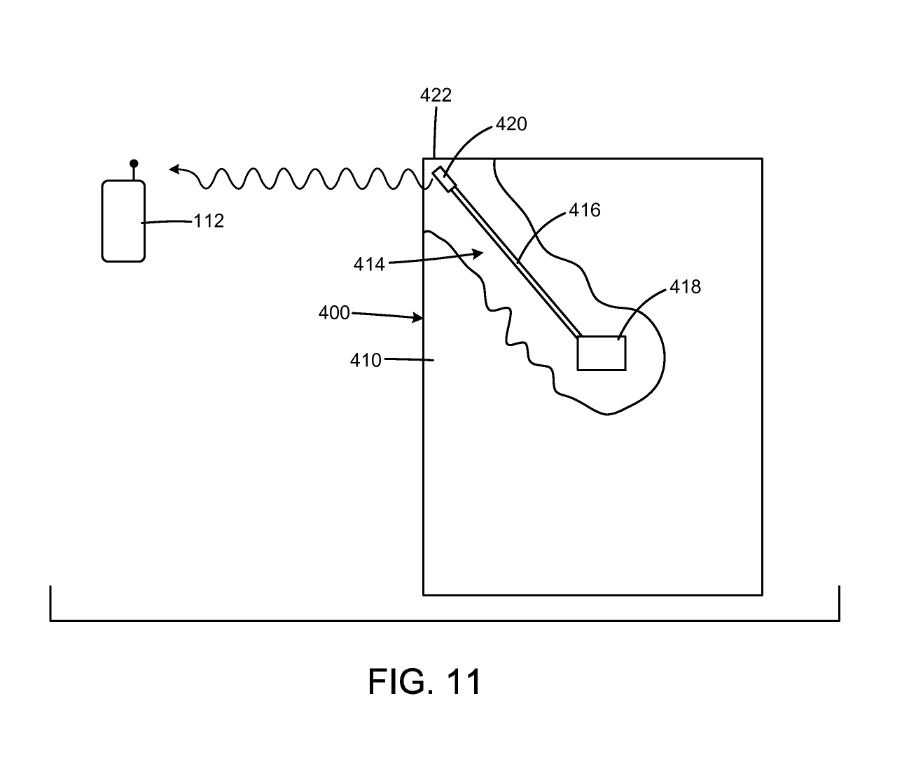

FIG. 11 illustrates an absorbent article, specifically a bed pad 400, which includes the ionic fluid sensor 414 of the present invention, which makes the bed pad operable for use with the radiofrequency transmitter 142 and the radiofrequency receiver/reader 112 of the present invention. More particularly, the portion of the bed pad that contacts a user is facing the viewer. Portions of the top layer 410 of the bed pad are cut away to more clearly show the underlying structure of the sensor 414. The bed pad sensor is embedded in the bed pad. The sensor comprises a radiofrequency sensor head 420, which is located near an edge 422 of the bed pad. A wicking channel 416 is installed from the sensor head to a pad of wicking material 418 located within the area of the bed pad that is most likely to get wet. When the pad of wicking material becomes wet, the wicking channel wicks the fluid from the pad of wicking material to the sensor head, which subsequently detects the presence of an ionic fluid. The sensor head is substantially identical to the ionic fluid sensor 122, with a sensor pad in fluid communication with the wicking channel.

In the context of the specification, the terms “absorbent article,” “disposable,” and “diaper” have the following definitions: “absorbent article” refers to devices that absorb and contain liquid, and more specifically, refers to devices that are placed against or in proximity to the body of the wearer to absorb and contain the various exudates discharged from the body. Absorbent articles include but are not limited to diapers, adult incontinent briefs, training pants, diaper holders and liners, sanitary napkins, bandages, bed pads, and the like. Absorbent articles also include wipes, such as household cleaning wipes, baby wipes, and the like. “Disposable” is used herein to describe articles that are generally not intended to be laundered or otherwise restored or reused i.e., they are intended to be discarded after a single use and, preferably, to be recycled, composted or otherwise disposed of in an environmentally compatible manner. “Diaper” refers to an absorbent article generally worn by infants and incontinent persons about the lower torso.

While a current embodiment of an ionic fluid detection system for absorbent articles has been described in detail, it should be apparent that modifications and variations thereto are possible, all of which fall within the true spirit and scope of the invention. For example, although a sensor that responds when it has not contacted an ionic fluid, and stops responding when shorted out has been described, the sensor can also have the antennae geometry assembled with a salt/salt-like conductive material, which becomes conductive when wet. Such a device could be constructed of a salt impregnated wicking material. In the presence of a liquid, such as water, a conductive media is formed, and allows the sensor to respond to radiofrequency interrogation. In this circumstance, the receiver/reader would transmit Wi-Fi signals indicating the sensor is dry in the absence of radiofrequency output from the sensor, and transmit Wi-Fi signals indicating the sensor is wet upon receiving radiofrequency output from the sensor. In both types of sensor, the sensor requires no energy source other than the interrogating radiofrequency wave, and no electronics other than the conditioning diode, which may be ink-jet fabricated, or is of a press-on type. Furthermore, the radiofrequency transmitter and the receiver/reader could be incorporated into a single device instead of the separate devices described. In addition, although building the sensor directly into the diapers been described, the sensor could also be applied to the diaper via an adhesive tape, glue, or other suitable fastening means. And although detection of the presence of an ionic fluid has been described, the ionic fluid detection system of the present invention can also be used as a proximity sensor. Once the person (child or adult patient) moves out of the range of the radiofrequency receiver/reader, an alarm similar to the wet diaper signal could be sent.

A. Geiger et al. 12

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An ionic fluid sensor comprising:
a radiofrequency receiver operable to receive a first radiofrequency signal;
a radiofrequency emitter connected to the receiver and operable to emit a second radiofrequency signal;
the emitter being responsive to the receiver to emit a second radiofrequency signal when the receiver receives the first radiofrequency signal;
the responsiveness of the emitter being enabled when the sensor is in a first wetness condition selected from the group of conditions including “wet” and “dry” and being disabled when in the other wetness condition.

2. The sensor of claim 1 wherein the second radiofrequency signal has a different frequency and wavelength than the first radiofrequency signal.

3. The sensor of claim 2 wherein the radiofrequency receiver is electrically connected to the radiofrequency emitter by a diode to form a resonant antennae pair.

4. The sensor of claim 1 further comprising:
a ground plane; and
a nonconductive, ionic fluid wicking material connected to the ground plane and at least one of the group consisting of the radiofrequency receiver and the radiofrequency emitter.

5. The sensor of claim 1 wherein the radiofrequency receiver includes an antenna having a length, the radiofrequency emitter includes an antenna having a length, and the lengths of the antennas are different.

6. The sensor of claim 5 wherein the length of the receiver antenna is greater than the length of the transmitter antenna.

7. The sensor of claim 3 wherein the diode multiplies the frequency from the radiofrequency receiver to the radiofrequency transmitter.

8. The sensor of claim 4 wherein upon contacting an ionic fluid, the nonconductive, ionic fluid wicking material becomes electrically conductive and shorts out at least one of the group consisting of the radiofrequency receiver and the radiofrequency emitter to the ground plane, thereby placing the sensor in the “wet” condition.

9. The sensor of claim 4 wherein in the absence of contact of the nonconductive, ionic fluid wicking material with an ionic fluid, the sensor is in the “dry” condition.

10. The sensor of claim 1 further comprising an absorbent article attached to the sensor.

11. The sensor of claim 10 wherein the absorbent article is selected from the group consisting of diapers, bandages, and bed pads.

12. The sensor of claim 10 wherein the absorbent article is attached to the sensor such that an ionic fluid contacting the absorbent article is transferred by the absorbent article into contact with the sensor.

13. A method of detecting wetness in an absorbent article in fluid communication with a user, the method comprising:
transmitting a radiofrequency activating signal in proximity to the article;
receiving the activating signal;
determining whether the article is in a dry condition or a wet condition;
in response to receiving the activating signal, generating a radiofrequency response signal in a first wetness condition selected from the group of conditions including “wet” and “dry” and not generating a radiofrequency response signal when in the other wetness condition.

14. The method of claim 13 wherein the radiofrequency response signal is not generated if the article is in the “wet” condition.

15. The method of claim 13 wherein the absorbent article is selected from the group consisting of diapers, bandages, and bed pads.

16. An ionic fluid sensor comprising:
a radiofrequency receiver operable to receive a first radiofrequency signal;
a radiofrequency emitter connected to the receiver and operable to emit a second radiofrequency signal;
the emitter being responsive to the receiver to emit a second radiofrequency signal when the receiver receives the first radiofrequency signal;
the responsiveness of the emitter being enabled when the sensor is in a first operational condition selected from the group of conditions including “activated” and “deactivated” and being disabled when in the other operational condition; and
the responsiveness of the emitter being enabled when the sensor is in both an “activated” condition and in a first wetness condition selected from the group of conditions including “wet” and “dry” and being disabled when in the other wetness condition.

17. The sensor of claim 16 wherein the sensor is placed in a “deactivated” operational condition when the emitter is shorted out to a ground plane by a removable conductive material contacting the emitter and the ground plane.

18. The sensor of claim 17 wherein the sensor is placed in an “activated” operational condition by removal of the conductive material from contact with at least one of the emitter and the ground plane.

19. The sensor of claim 17 wherein the sensor is placed in an “activated” operational condition by breakage of the conductive material to terminate electrical contact between the emitter and the ground plane.

20. The sensor of claim 17 wherein the sensor is placed in an “activated” operational condition only immediately prior to placing the sensor on a wearer.
